(19) United States
(12) Patent Application Publication
(10) Pub. No.: US 2024/0395406 A1
(43) Pub. Date: Nov. 28, 2024

(54) LEARNING APPARATUS, ANALYSIS APPARATUS, LEARNING METHOD, ANALYSIS METHOD, AND PROGRAM

(71) Applicants: NIPPON TELEGRAPH AND TELEPHONE CORPORATION, Tokyo (JP); NTT Research Inc., Sunnyvale, CA (US)

(72) Inventors: Ryohei SHIBUE, Musashino-shi (JP); Kunio KASHINO, Musashino-shi (JP); Masahiro NAKANO, Musashino-shi (JP); Hitonobu TOMOIKE, Fuchu-shi (JP)

(73) Assignees: NIPPON TELEGRAPH AND TELEPHONE CORPORATION, Tokyo (JP); NTT Research Inc., Sunnyvale, CA (US)

(21) Appl. No.: 18/695,959

(22) PCT Filed: Oct. 8, 2021
(86) PCT No.: PCT/US2021/054086

§ 371 Date: Mar. 27, 2024

(51) Int. Cl. G16H 50/20

(52) U.S. Cl. CPC G16H 50/20

(57) ABSTRACT

An aspect of the present invention is a learning apparatus including a time series acquisition unit that is configured to, with a time series of an amplitude of a fluctuating oscillator whose amplitude changes periodically being defined as an oscillator time series, acquire an observed time series which is a time series represented by an oscillator linear sum which is a linear sum of the oscillator time series and a learning processing execution unit that is configured to use an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, wherein the learning processing execution unit is configured to update the linear sum estimation learning model on the basis of a result of execution of the linear sum estimation learning model.

### TECHNICAL FIELD

The present invention relates to a learning apparatus, an analysis apparatus, a learning method, an analysis method, and program.

### BACKGROUND ART

Cardiac sounds are an important clue for knowing the condition of a patient's circulatory system.

### CITATION LIST

### Non-Patent Literature

### [Non-Patent Document 1]

L G Gamero and R Watrous, “Detection of the first and second heart sound using probabilistic models,” Proc. IEEE Eng. Med. Biol. Soc., vol. 3, pp. 2877-2880, 2003.

### [Non-Patent Document 2]

D Gill, N Gavrieli, and N Intrator, “Detection and identification of heart sounds using homomorphic envelogram and self-organizing probabilistic model,” pp. 957-960, 2005.

### [Non-Patent Document 3]

A D Ricke, R J Povinelli, and M T Johnson, “Automatic segmentation of heart sound signals using hidden Markov models,” pp. 953-956, 2005.

### [Non-Patent Document 4]

P Sedighian, A W Subudhi, F Scalzo, and S Asgari, “Pediatric heart sound segmentation using hidden Markov model,” pp. 5490-5493, 2014.

### [Non-Patent Document 5]

S E Schmidt, C Holst-Hansen, C Graff, E Toft, and J J Struijk, “Segmentation of heart sound recordings by a duration-dependent hidden Markov model,” Physiol. Meas., vol. 31, no. 4, pp. 513-529, 2010.

### [Non-Patent Document 6]

D B Springer, L Tarassenko, and G D Clifford, “Logistic regression-HSMM-based heart sound segmentation,” IEEE Trans. Biomed. Eng., vol. 63, no. 4, pp. 822-832, 2016.

### [Non-Patent Document 7]

S Ari and Go Saha, “Classification of heart sounds using empirical mode decomposition based features,” Int. J. Med. Eng. Inform., vol. 1, no. 1, pp. 91-108, 2008.

### [Non-Patent Document 8]

H Sun, W Chen, and J Gong, “An improved empirical mode decomposition-wavelet algorithm for phonocardiogram signal denoising and its application in the first and second heart sound extraction,” pp. 187-191, 2013.

### [Non-Patent Document 9]

C D Papadaniil and L J Hadjileontiadis, “Efficient heart sound segmentation and extraction using ensemble empirical mode decomposition and kurtosis features,” IEEE J Biomed Health Inform, vol. 18, no. 4, pp. 1138-1152, 2014.

### [Non-Patent Document 10]

E F Blick, H N Sabbah, and P D Stein, “One-dimensional model of diastolic semilunar valve vibrations productive of heart sounds,” J. Biomech., vol. 12, no. 3, pp. 223-227, 1979.

### [Non-Patent Document 11]

M J Johnson and A S Willsky, “Stochastic variational inference for bayesian time series models,” pp. II-1854-II-1862, 2014.

### [Non-Patent Document 12]

D L Sikarskie, P D Stein, and M Vable, “A mathematical model of aortic valve vibration,” J. Biomech., vol. 17, no. 11, pp. 831-837, 1984.

### [Non-Patent Document 13]

H Ozcan Gulcur and Y Bahadirlar, “Estimation of systolic blood pressure from the second heart sounds,” Int. Biom. Eng. Days, pp. 39-41, 1998.

### [Non-Patent Document 14]

X-Y Zhang and Y-T Zhang, “Model-based analysis of effects of systolic blood pressure on frequency characteristics of the second heart sound,” Proc. IEEE Eng. Med. Biol. Soc., vol. 2006, pp. 2888-2891, 2006.

### [Non-Patent Document 15]

T Matsuda and F Komaki, “Multivariate time series decomposition into oscillation components,” Neural Comput., vol. 29, no. 8, pp. 2055-2075, 2017.

### [Non-Patent Document 16]

H Soulat, E P Stephen, A M Beck, and P L Purdon, “State space methods for phase amplitude coupling analysis,” bioRxiv, 2019.

### [Non-Patent Document 17]

E Archer, I M Park, L Buesing, J Cunningham, and L Paninski, “Black box variational inference for state space models,” 2015.

### SUMMARY OF INVENTION

### Technical Problem

Although a plurality of previous studies have proposed methods of automatically estimating the condition of a heart at each time from a cardiac sound time series, the accuracy of estimating the condition of the heart at each time is not good. For this reason, even now, estimating the condition of the heart through cardiac sound requires labor to create supervised data using an electrocardiograph in advance.

The poor accuracy of estimating the condition of the heart at each time will be described in a little more detail. A waveform indicated by a time series of a cardiac sound is decomposed into amplitude waveforms of a plurality of oscillators whose amplitudes change periodically. That is, the waveform of the time series of the cardiac sound is a linear sum of amplitude waveforms of a plurality of fluctuating oscillators. A fluctuating oscillator is an oscillator whose amplitude changes periodically. The poor accuracy of estimating the condition of the heart at each time means specifically that the accuracy of decomposing the time series of the cardiac sound into a linear sum of time series of the amplitudes of the fluctuating oscillators is not good.

Meanwhile, such a problem is not necessarily limited to cardiac sounds, and also exists for analysis of a time series represented by a linear sum of amplitude waveforms of the fluctuating oscillators.

The present invention was contrived in view of such circumstances, and an object thereof is to provide a technique of improving the accuracy of analysis of a time series represented by a linear sum of amplitude waveforms of a fluctuating oscillator—whose amplitude changes periodically.

### Solution to Problem

According to an aspect of the present invention, there is provided a learning apparatus including: a time series acquisition unit that is configured to, with a time series of an amplitude of a fluctuating oscillator whose amplitude changes periodically being defined as an oscillator time series, acquire an observed time series which is a time series represented by an oscillator linear sum which is a linear sum of the oscillator time series; and a learning processing execution unit that is configured to use an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, wherein the learning processing execution unit is configured to update the linear sum estimation learning model on the basis of a result of execution of the linear sum estimation learning model.

According to an aspect of the present invention, there is provided an analysis apparatus including: an analysis target acquisition unit that is configured to acquire a time series which is a target for analysis; and an analysis unit that is configured to analyze a time series which is a target for analysis using a learned linear sum estimation learning model obtained by a learning apparatus including a time series acquisition unit that is configured to, with a time series of an amplitude of a fluctuating oscillator whose amplitude changes periodically being defined as an oscillator time series, acquire an observed time series which is a time series represented by an oscillator linear sum which is a linear sum of the oscillator time series and a learning processing execution unit that is configured to use an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, the learning processing execution unit updating the linear sum estimation learning model on the basis of a result of execution of the linear sum estimation learning model.

According to an aspect of the present invention, there is provided a learning method including: a time series acquisition step of, with a time series of an amplitude of a fluctuating oscillator whose amplitude changes periodically being defined as an oscillator time series, acquiring an observed time series which is a time series represented by an oscillator linear sum which is a linear sum of the oscillator time series; and a learning process execution step of using an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, wherein the learning process execution step includes updating the linear sum estimation learning model on the basis of a result of execution of the linear sum estimation learning model.

According to an aspect of the present invention, there is provided an analysis method including: an analysis target acquisition step of acquiring a time series which is a target for analysis; and an analysis step of analyzing a time series which is a target for analysis using a learned linear sum estimation learning model obtained by a learning apparatus including a time series acquisition unit that is configured to, with a time series of an amplitude of a fluctuating oscillator whose amplitude changes periodically being defined as an oscillator time series, acquire an observed time series which is a time series represented by an oscillator linear sum which is a linear sum of the oscillator time series and a learning processing execution unit that is configured to use an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, the learning processing execution unit updating the linear sum estimation learning model on the basis of a result of execution of the linear sum estimation learning model.

According to an aspect of the present invention, there is provided a computer program for causing a computer to function as the above learning apparatus.

According to an aspect of the present invention, there is provided a computer program for causing a computer to function as the above analysis apparatus.

### Advantageous Effects of Invention

According to the present invention, it is possible to improve the accuracy of analysis of a time series represented by a linear sum of amplitude waveforms of a fluctuating oscillator whose amplitude changes periodically.

### BRIEF DESCRIPTION OF DRAWINGS

FIG. 1 is a diagram illustrating an example of a configuration of an analysis system of an embodiment.

FIG. 2 is a diagram illustrating an example of a hardware configuration of a learning apparatus in the embodiment.

FIG. 3 is a diagram illustrating an example of a configuration of a control unit included in the learning apparatus in the embodiment.

FIG. 4 is a flow chart illustrating an example of a flow of processes which are executed by the learning apparatus in the embodiment.

FIG. 5 is a diagram illustrating an example of a hardware configuration of an analysis apparatus in the embodiment.

FIG. 6 is a diagram illustrating an example of a configuration of a control unit included in the analysis apparatus in the embodiment.

FIG. 7 is a flow chart illustrating an example of a flow of processes which are executed by the analysis apparatus in the embodiment.

FIG. 8 is a first diagram illustrating an example of a result of an experiment using the analysis system of the embodiment.

FIG. 9 is a second diagram illustrating an example of a result of an experiment using the analysis system of the embodiment.

FIG. 10 is a third diagram illustrating an example of a result of an experiment using the analysis system of the embodiment.

FIG. 11 is a fourth diagram illustrating an example of a result of an experiment using the analysis system of the embodiment.

FIG. 12 is a fifth diagram illustrating an example of a result of an experiment using the analysis system of the embodiment.

FIG. 13 is a sixth diagram illustrating an example of a result of an experiment using the analysis system of the embodiment.

FIG. 14 is a seventh diagram illustrating an example of a result of an experiment using the analysis system of the embodiment.

FIG. 15 is an eighth diagram illustrating an example of a result of an experiment using the analysis system of the embodiment.

### DESCRIPTION OF EMBODIMENTS

### Embodiment

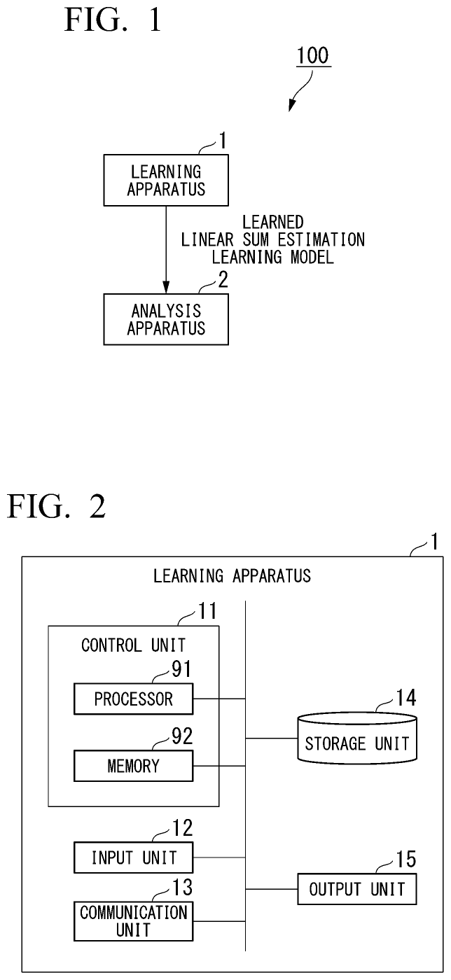

FIG. 1 is a diagram illustrating an example of a configuration of an analysis system 100 of an embodiment. Hereinafter, for the purpose of simplification of description, the analysis system 100 will be described with a case in which a target for analysis in the analysis system 100 is a time series of a cardiac sound (hereinafter referred to as a “cardiac sound time series”) and the analysis system 100 estimates the condition of a heart which is a generation source of a cardiac sound time series on the basis of the cardiac sound time series as an example.

However, a time series which is a target for analysis in the analysis system 100 does not necessarily have to be a time series of a cardiac sound. The time series which is a target for analysis in the analysis system 100 may be any time series insofar as it is a time series represented by a linear sum of amplitude waveforms of an oscillator whose amplitude changes periodically (hereinafter referred to as a “fluctuating oscillator”). Since the waveform is a time series, the linear sum of amplitude waveforms of a fluctuating oscillator is a linear sum of an amplitude time series of a fluctuating oscillator. Hereinafter, an amplitude time series of a fluctuating oscillator is referred to as “oscillator time series”. Hereinafter, a linear sum of oscillator time series is referred to as “oscillator linear sum”.

In addition, the result of analysis which is output by the analysis system 100 does not necessarily have to be the result of estimation of the condition of a heart. The result of analysis which is output by the analysis system 100 may be any result insofar as it is a result obtained on the basis of the oscillator linear sum. The result of analysis which is output by the analysis system 100 may be an amplitude time series of each fluctuating oscillator of the oscillator linear sum.

The analysis system 100 includes a learning apparatus 1 and an analysis apparatus 2. The learning apparatus 1 updates a machine learning model that estimates an oscillator linear sum (hereinafter referred to as a “linear sum estimation learning model”) through learning on the basis of an input cardiac sound time series.

Meanwhile, the machine learning model is a mathematical model including one or a plurality of processes in which the condition and order of execution (hereinafter referred to as an “execution rule”) are determined in advance. The term “learning” refers to updating a machine learning model using a machine learning method. In addition, updating a machine learning model involves appropriately adjusting values of predetermined parameters included in the machine learning model. In addition, the execution of a machine learning model involves executing each process included in the machine learning model in accordance with the execution rule.

Meanwhile, the machine learning model is represented by, for example, a neural network. Meanwhile, the neural network is a circuit such as an electronic circuit, an electrical circuit, an optical circuit, or an integrated circuit and is a circuit that represents a machine learning model. Updating a machine learning model means also to update a neural network that represents the machine learning model by learning. Updating a neural network by learning means to update values of parameters of the neural network. In addition, the parameters of a neural network are parameters of a circuit constituting the neural network, and are also parameters of a learning model represented by the circuit constituting the neural network.

The neural network that represents a linear sum estimation learning model may be any neural network insofar as it is a neural network capable of representing the linear sum estimation learning model. The neural network that represents a linear sum estimation learning model is, for example, a deep neural network.

The learning apparatus 1 learns a linear sum estimation learning model until a predetermined end condition (hereinafter referred to as a “learning end condition”) is satisfied. The learning end condition is, for example, a condition that learning has been performed a predetermined number of times. The learning end condition may be, for example, a condition that a change of a linear sum estimation learning model due to update is smaller than a predetermined change.

The analysis apparatus 2 uses a learned linear sum estimation learning model obtained by the learning apparatus 1 to estimate an oscillator linear sum indicating an input cardiac sound time series. The learned linear sum estimation learning model is a linear sum estimation learning model at a timing when the learning end condition is satisfied.

### <Linear Sum Estimation Learning Model and Process of Learning of Linear Sum Estimation Learning Model>

The relationship between a linear sum estimation learning model and a process in which the learning apparatus 1 learns the linear sum estimation learning model (hereinafter referred to as a “learning process”) will be described. The linear sum estimation learning model includes a machine learning model that estimates the posterior distribution of the state of a cardiac cycle on the basis of the input cardiac sound time series. Hereinafter, a machine learning model that estimates the posterior distribution of the state of a cardiac cycle on the basis of the input cardiac sound time series is referred to as a “cardiac cycle state posterior distribution learning model”. The linear sum estimation learning model includes a machine learning model that estimates the posterior distribution of an oscillator time series on the basis of the posterior distribution of the state of a cardiac cycle. Hereinafter, a machine learning model that estimates the posterior distribution of an oscillator time series on the basis of the posterior distribution of the state of a cardiac cycle is referred to as an “oscillator time series posterior distribution learning model” The linear sum estimation learning model includes a machine learning model that estimates the marginal distribution of a cardiac sound time series using the posterior distribution of an oscillator time series. Hereinafter, a machine learning model that estimates the marginal distribution of a cardiac sound time series using the posterior distribution of an oscillator time series is referred to as a “cardiac sound time series marginal distribution learning model”.

The learning process includes a time series input process, a cardiac cycle state posterior distribution estimation process, an oscillator time series posterior distribution estimation process, a cardiac sound time series marginal distribution estimation process, and an update process.

The time series input process is a process in which a cardiac sound time series is input to a linear sum estimation learning model.

The cardiac cycle state posterior distribution estimation process is a process of estimating the posterior distribution of the state of a cardiac cycle on the basis of the input cardiac sound time series by the execution of a cardiac cycle state posterior distribution learning model.

The oscillator time series posterior distribution estimation process is a process of estimating the posterior distribution of an oscillator time series on the basis of the posterior distribution of the state of a cardiac cycle by the execution of an oscillator time series posterior distribution learning model.

The cardiac sound time series marginal distribution estimation process is a process of estimating the marginal distribution of a cardiac sound time series using the posterior distribution of an oscillator time series by the execution of a cardiac sound time series marginal distribution learning model.

The update process is a process of updating a linear sum estimation learning model so that a cardiac sound time series which is input through the time series input process increases a marginal likelihood based on the marginal distribution estimated in the cardiac sound time series marginal distribution estimation process.

Updating a linear sum estimation learning model involves, more specifically, updating a cardiac cycle state posterior distribution learning model, an oscillator time series posterior distribution learning model, and a cardiac sound time series marginal distribution learning model.

### <Cardiac Cycle State Posterior Distribution Learning Model>

The cardiac cycle state posterior distribution learning model may be any mathematical model insofar as the posterior distribution of the state of a cardiac cycle can be calculated on the basis of a cardiac sound time series. States of a cardiac cycle include four states, that is, an S1 sound, a systolic phase, an S2 sound, and a diastolic phase. The four states of the cardiac cycle, that is, an S1 sound, a systolic phase, an S2 sound, and a diastolic phase are periodically repeated. More specifically, the state of the cardiac cycle transitions from the state of the S1 sound to the state of the systolic phase. The state of the cardiac cycle transitions from the state of the systolic phase to the state of the S2 sound. The state of the cardiac cycle transitions from the state of the S2 sound to the state of the diastolic phase. The state of the cardiac cycle transitions from the state of the diastolic phase to the state of the S1 sound.

The sound in the state of the S1 sound and the sound in the state of the S2 sound are extremely loud sounds among cardiac sounds, and are sounds caused by the oscillation of valves which are generated when the mitral valve and the aortic valve are closed. Therefore, the cardiac sound time series is a time series in which the amplitudes of a nonlinear oscillator mainly equivalent to the oscillation of an intracardiac valve are added to each other while changing periodically.

The cardiac cycle state posterior distribution learning model is, for example, a mathematical model that acquires a posterior probability distribution which is represented by the following Expression (1) on the basis of the cardiac sound time series. The cardiac sound time series is represented by the following Expression (2).

$$\begin{matrix}[ Expression 1 ] & \\ p ( {\overline{z}}_{1 : R} \vert {x}_{1 : R} ) = \frac{p ( {\overline{z}}_{1} ) {\prod }_{r = 2}^{R} p ( {\overline{z}}_{r} \vert {\overline{z}}_{r - 1} ) p ( {x}_{1 : R} \vert {\overline{z}}_{1 : R} )}{{\sum }_{{\overline{z}}_{1 : R}} p ( {\overline{z}}_{1} ) {\prod }_{r = 2}^{R} p ( {\overline{z}}_{r} \vert {\overline{z}}_{r - 1} ) p ( {x}_{1 : R} \vert {\overline{z}}_{1 : R} )} & ( 1 )\end{matrix}$$

$$\begin{matrix}[ Expression 2 ] & \\ {x}_{1 : R} = {\{ {x}_{r} \}}_{r = 1}^{R} & ( 2 )\end{matrix}$$

$$\begin{matrix}[ Expression 3 ] & \\ {x}_{r} ∈ {ℝ}^{J} , r = 1 , 2 , … , R & ( 3 )\end{matrix}$$

$$[ Expression 4 ]$$

$${z}_{r} ∈ 1 , 2 , 3 , 4 \qquad ( 4 )$$

$$[ Expression 5 ]$$

$${\overline{z}}_{r} = ( {z}_{r} , {e}_{r} ) \qquad ( 5 )$$

$$\begin{matrix}[ Expression 6 ] & \\ {e}_{r} ∈ \{ 1 , ⋯ , m \} & ( 6 )\end{matrix}$$

J is the number of microphones that acquire a cardiac sound. R is the length of the cardiac sound time series. In addition, zᵣ is an unobserved state of the cardiac sound time series at the r-th time. The unobserved state is specifically the state of the cardiac cycle. Since there are four states of the cardiac cycle as described above, the possible values of zᵣ are 1 to 4 as shown in Expression (4).

The state of the cardiac cycle does not transition to the next state immediately after state transition occurs. After the state transition occurs, the post-transition state continues for a finite period of time. That is, in each state of the cardiac cycle, there is a finite duration until the transition to the next state. Consequently, the transition probability defined by the following Expression (7) is defined.

$$\begin{matrix}[ Expression 7 ] & \\ p ( {z}_{r : r + δ - 1} = j , {z}_{r + δ} \neq j \vert {z}_{r} = i ) = {A}_{ij} {p}_{j} ( δ ) , & ( 7 )\end{matrix}$$

$$[ Expression 8 ]$$

$$A = [ [ 0 , 1 , 0 , 0 ] , [ 0 , 0 , 1 , 0 ] , [ 0 , 0 , 0 , 1 ] , [ 1 , 0 , 0 , 0 ] ] \qquad ( 8 )$$

A in Expression (8) is a transition matrix. In addition, pⱼ(δ) is the distribution of duration. The distribution of duration represented by pⱼ(δ) is represented by, for example, the negative binomial distribution of the following Expression (9).

$$\begin{matrix}[ Expression 9 ] & \\ {p}_{i} ( δ ) = ( \begin{matrix}δ + m - 2 \\ δ - 1\end{matrix} ) {( 1 - {θ}_{i} )}^{m} {θ}_{i}^{δ - 1} , δ = 1 , 2 , … , & ( 9 )\end{matrix}$$

Here, θⱼ is the probability of success of a trial, and m is a shape parameter. In addition, eᵣ in Expression (6) is a pseudo-state. The pseudo-state is an amount indicating how long each state has continued. zᵣ\hat{ }{-} which is defined by Expression (5) using the pseudo-states eᵣ and zᵣ. follows a Markov process with a probability transition matrix which is determined by (7), (8) and (9). Meanwhile, hereinafter, a symbol with a bar above the symbol is represented as symbol\hat{ }{-}. For example, zᵣ\hat{ }{-} indicates a symbol with a bar above the symbol zᵣ. Therefore, zᵣ\hat{ }{-} indicates the symbol on the left side of Expression (5). In addition, as in Expression (10), the probability of zᵣ coincides with the probability of marginalizing zᵣ\hat{ }{-} with respect to the pseudo-state.

$$\begin{matrix}[ Expression 10 ] & \\ p ( {z}_{1 : R} ) = {\sum }_{{c}_{r}} p ( {\{ ( {z}_{r} , {e}_{r} ) \}}_{r = 1}^{R} ) = {\sum }_{{e}_{r}} p ( {\overline{z}}_{1 : R} ) & ( 10 )\end{matrix}$$

In the calculation of Expression (1), it is necessary to calculate the numerator of Expression (1) for all combinations of latent variables z\hat{ }{-} and then perform normalization so that the posterior probabilities on the left side add up to 1. However, the number of possible combinations of states is (4m)^{R}, and the amount of calculation is on the order of (4m)^{R}. Such an increase in the amount of calculation is suppressed by, for example, replacing the term of the following Expression (11) in the numerator of Expression (1) with the approximate expression on the right side of Expression (13) which is an expression using the function of Expression (12). Hereinafter, the function of Expression (12) is referred to as a “potential”.

$$\begin{matrix}[ Expression 11 ] & \\ p ( {x}_{1 : R} \vert {\overline{z}}_{1 : R} ) & ( 11 )\end{matrix}$$

$$\begin{matrix}[ Expression 12 ] & \\ ψ ( {\overline{z}}_{r} , {x}_{1 : R} ) & ( 12 )\end{matrix}$$

$$\begin{matrix}[ Expression 13 ] & \\ p ( {x}_{1 : R} \vert {\overline{z}}_{1 : R} ) \approx {\prod }_{r = 1}^{R} ψ ( {\overline{z}}_{r} , {x}_{1 : R} ) & ( 13 )\end{matrix}$$

By the approximation of Expression (13), the amount of calculation for calculating the value of Expression (1) can be reduced to the order of 4mR. Meanwhile, the approximate expression of the above (13) is an example, and the potential may be any function insofar as it is a part of the function representing the posterior probability distribution of the state of the cardiac cycle and a function in which content to be represented is defined in advance.

An expression indicating the potential is obtained using a machine learning model such as, for example, a neural network on the basis of the cardiac sound time series. Therefore, in a case where the posterior probability distribution of the state of the cardiac cycle is represented using the potential, the expression indicating the potential is also updated in learning of the linear sum estimation learning model.

### <Oscillator Time Series Posterior Distribution Learning Model>

The oscillator time series posterior distribution learning model will be described in more detail. The oscillator time series posterior distribution learning model is a mathematical model representing the relationship between a probabilistic state transition such as a hidden Markov model or a hidden semi-Markov model and a symbol output that is information which is probabilistically output in each state. The state in the mathematical model representing the relationship between a probabilistic state transition and a symbol output is the state of the cardiac cycle.

The symbol output in the mathematical model representing the relationship between a probabilistic state transition and a symbol output is an oscillator time series. In a case where the mathematical model representing the relationship between a probabilistic state transition and a symbol output is a hidden semi-Markov model, the mathematical model also represents the duration of each state.

### <Cardiac Sound Time Series Marginal Distribution Learning Model>

The cardiac sound time series marginal distribution learning model will be described in more detail. The cardiac sound time series is observed as the linear sum of the fluctuating oscillators in accordance with Expression (14). Expression (19) is a distribution followed by the fluctuating oscillator and an expression derived from Expression (17) representing the generation mechanism of the cardiac sound time series. Expression (17) is a differential equation representing the generation mechanism of the cardiac sound time series.

$$\begin{matrix}[ Expression 14 ] & \\ {x}_{r} = ( \begin{matrix}1 & 0 & ⋯ & 1 & 0 \\ {g}_{21}^{( 1 )} & {g}_{21}^{( 2 )} & ⋯ & {g}_{2 L}^{( 1 )} & {g}_{2 L}^{( 2 )} \\ ⋮ & & ⋱ & & ⋮ \\ {g}_{J 1}^{( 1 )} & {g}_{J 1}^{( 2 )} & ⋯ & {g}_{JL}^{( 1 )} & {g}_{JL}^{( 2 )}\end{matrix} ) ( \begin{matrix}{y}_{r , 1}^{( 1 )} \\ {y}_{r , 1}^{( 2 )} \\ ⋮ \\ {y}_{r , L}^{( 1 )} \\ {y}_{r , L}^{( 2 )}\end{matrix} ) + {w}_{r} , & ( 14 )\end{matrix}$$

$$\begin{matrix}[ Expression 15 ] & \\ {w}_{r} ∼ N ( 0 , {τ}^{2} I ) & ( 15 )\end{matrix}$$

$$\begin{matrix}[ Expression 16 ] & \\ {y}_{rl} = ( {y}_{rl}^{( 1 )} , {y}_{rl}^{( 2 )} ) ∈ {ℝ}^{2} & ( 16 )\end{matrix}$$

$$\begin{matrix}[ Expression 17 ] & \\ M \ddot{u} = D \dot{u} + Ku = Δ P & ( 17 )\end{matrix}$$

Here, τ is the variance of an observation noise distribution. The amount yrl on the left side of Expression (16) indicates the amplitude of the 1-th fluctuation oscillator at a time r.

M is the mass of the valve of the heart. In addition, u is the displacement of the valve of the heart. ΔP is pressure exerted on the valve of the heart. D is an attenuation coefficient indicating the magnitude of movement attenuation of the valve of the heart. K is the stiffness coefficient of the valve of the heart.

The formal solution of Expression (17) is the following Expression (18).

$$\begin{matrix}[ Expression 18 ] & \\ u ( t ) = C exp ( - α t ) sin ( ω t - ψ ) & ( 18 )\end{matrix}$$

C and α are constants. In addition, ω is an angular frequency, ψ is an initial phase shift, and t is a time.

Since the amplitude of the waveform indicated by the cardiac sound time series is proportional to the first-order differential of displacement u of the valve of the heart (that is, the speed of the heart valve membrane), the fluctuating oscillator is represented by a second-order autoregressive model. Therefore, the following Expression (19) is established.

$$\begin{matrix}[ Expression 19 ] & \\ ( \begin{matrix}{y}_{rl}^{( 1 )} \\ {y}_{rl}^{( 2 )}\end{matrix} ) = {a}_{l} ( \begin{matrix}cos ( \frac{2 π {f}_{l}}{{f}_{s}} ) & sin ( \frac{2 π {f}_{l}}{{f}_{s}} ) \\ - sin ( \frac{2 π {f}_{l}}{{f}_{s}} ) & cos ( \frac{2 π {f}_{l}}{{f}_{s}} )\end{matrix} ) ( \begin{matrix}{y}_{r - 1 , l}^{( 1 )} \\ {y}_{r - 1 , l}^{( 2 )}\end{matrix} ) + ( \begin{matrix}{v}_{rli}^{( 1 )} \\ {v}_{rli}^{( 2 )}\end{matrix} ) , ( \begin{matrix}{v}_{rli}^{( 1 )} \\ {v}_{rli}^{( 2 )}\end{matrix} ) ∼ N ( 0 , {σ}_{li} 2 I ) , & ( 19 )\end{matrix}$$

Here, aₗ is the attenuation coefficient of the l-th fluctuation oscillator, fₗ is the average frequency of the l-th fluctuation oscillator, fₛ is a sampling frequency, and σₗᵢ² is the variance of a system noise distribution. The variance σₗᵢ² strongly depends on the state of the cardiac cycle, and changes a dominant fluctuating oscillator included in the cardiac sound time series on the basis of the state of the cardiac cycle. Particularly, a large value of the variance σₗᵢ² means that the 1-th fluctuation oscillator is dominant in a case where the state of the cardiac cycle is i.

### <Update Process>

The update process will be described in more detail. In the update process, for example, the linear sum estimation learning model is updated so as to maximize a log marginal likelihood represented by the following Expression (20).

$$\begin{matrix}[ Expression 20 ] & \\ log p ( {x}_{1 : R} ) & ( 20 )\end{matrix}$$

In the update process, for example, the linear sum estimation learning model may be updated so as to maximize the lower bound of the likelihood represented by the following Expression (21). Meanwhile, updating the linear sum estimation learning model means specifically updating, for example, {θ-j}, {aₗ}, {fₗ}, {σₗᵢ}, {gₗᵢ} and τ.

$$\begin{matrix}[ Expression 21 ] & \\ {E}_{q ( {y}_{1 : R} , {\overline{z}}_{1 : R} )} [ log \frac{p ( {x}_{1 : R} \vert {y}_{1 : R} ) p ( {y}_{1 : R} \vert {\overline{z}}_{1 : R} ) p ( {\overline{z}}_{1 : R} )}{q ( {y}_{1 : R} , {\overline{z}}_{1 : R} )} ] & ( 21 )\end{matrix}$$

The log marginal likelihood represented by Expression (20) and the lower bound of the likelihood represented by Expression (21) are related to the following Expression (22).

$$\begin{matrix}[ Expression 22 ] & \\ log p ; ( {x}_{1 : R} ) \geq {E}_{q ( {y}_{1 : R} , {\overline{z}}_{1 : R} )} [ log \frac{p ( {x}_{1 : R} \vert {y}_{1 : R} ) p ( {y}_{1 : R} \vert {\overline{z}}_{1 : R} ) p ( {\overline{z}}_{1 : R} )}{q ( {y}_{1 : R} , {\overline{z}}_{1 : R} )} ] & ( 22 )\end{matrix}$$

The term of the following Expression (23) in Expression (22) represents Expression (14). The term of the following Expression (24) in Expression (22) represents Expression (19). The term of the following Expression (25) in Expression (22) is the transition probability of z\hat{ }{-} which is derived from Expressions (7), (8), (9), and (10).

$$\begin{matrix}[ Expression 23 ] & \\ p ( {x}_{1 : R} \vert {y}_{1 : R} ) & ( 23 )\end{matrix}$$

$$\begin{matrix}[ Expression 24 ] & \\ p ( {y}_{1 : R} \vert {\overline{z}}_{1 : R} ) & ( 24 )\end{matrix}$$

$$\begin{matrix}[ Expression 25 ] & \\ p ( {\overline{z}}_{1 : R} ) & ( 25 )\end{matrix}$$

In maximizing the lower bound of the likelihood represented by Expression (21), the relations of the following Expressions (26) and (27) are used in order to reduce the amount of calculation.

$$\begin{matrix}[ Expression 26 ] & \\ q ( {y}_{1 : R} , {\overline{z}}_{1 : R} ) = q ( {y}_{1 : R} ) q ( {\overline{z}}_{1 : R} ) & ( 26 )\end{matrix}$$

$$\begin{matrix}[ Expression 27 ] & \\ q ( {\overline{z}}_{1 : R} ) ∝ p ( {\overline{z}}_{1} ) {\prod }_{r = 2}^{R} p ( {\overline{z}}_{r} \vert {\overline{z}}_{r - 1} ) {\prod }_{r = 1}^{R} ψ ( {\overline{z}}_{r} , {x}_{1 : R} ) & ( 27 )\end{matrix}$$

The potential is included in Expression (27), and as described above, the expression indicating the potential is obtained by a machine learning model such as a neural network. The potential is, for example, the following Expression (28).

$$\begin{matrix}[ Expression 28 ] & \\ ψ ( {\overline{z}}_{r} = ( i , δ ) , {x}_{1 : R} ) = {[ ϕ ( {x}_{r - s + 1 : r} ) ]}_{( i , δ )} , & ( 28 )\end{matrix}$$

Here, φ(⋅) appearing on the right side of Expression (28) is a mapping from the following Expression (29) to the following Expression (30).

$$\begin{matrix}[ Expression 29 ] & \\ {x}_{r - s + 1 : r} ∈ {ℝ}^{sd} & ( 29 )\end{matrix}$$

$$\begin{matrix}[ Expression 30 ] & \\ ϕ ( {x}_{r - s + 1 : r} ) ∈ {ℝ}_{+}^{4 \times m} , & ( 30 )\end{matrix}$$

Here, s is a window length having a fixed length determined in advance. The symbol of the following Expression (31) appearing on the right side of Expression (28) is an operator for returning the (i, δ)-th element of an input matrix. Meanwhile, the input matrix is specifically (30) expression.

$$\begin{matrix}[ Expression 31 ] & \\ {[ \cdot ]}_{( i , δ )} & ( 31 )\end{matrix}$$

As the form of the expression indicating the potential, those used in supervised cardiac sound segmentation such as a convolutional neural network (CNN) or a recurrent neural network (RNN) may be used.

Meanwhile, the following Expression (32) and the following Expression (33) are a z\hat{ }{-} initial probability and a transition probability which are derived from Expressions (7), (8), (9), and (10).

$$\begin{matrix}[ Expression 32 ] & \\ p ( {\overline{z}}_{1} ) & ( 32 )\end{matrix}$$

$$\begin{matrix}[ Expression 33 ] & \\ p ( {\overline{z}}_{r} \vert {\overline{z}}_{r - 1} ) , r = 2 , … , R & ( 33 )\end{matrix}$$

The relation of Expression (22) is converted into the relation of the following Expression (34) by Expressions (23) to (33).

$$\begin{matrix}[ Expression 34 ] & \\ log p ( {x}_{1 : R} ) \geq log ρ ( {x}_{1 : R} ) + {E}_{q ( {\overline{z}}_{1 : R} )} [ log \frac{p ( {\overline{z}}_{1 : R} )}{q ( {\overline{z}}_{1 : R} )} ] & ( 34 )\end{matrix}$$

$$\begin{matrix}[ Expression 35 ] & \\ ρ ( {x}_{1 : R} ) = \int p ( {x}_{1 : R} \vert {y}_{1 : R} ) exp ( {E}_{q ( {\overline{z}}_{1 : R} )} [ log p ( {y}_{1 : R} \vert {\overline{z}}_{1 : R} ) ] ) {dy}_{1 : R} & ( 35 )\end{matrix}$$

By using a message propagation method, the lower bound of the likelihood represented by Expression (21) is maximized with the amount of calculation smaller than the amount of calculation required to maximize the log marginal likelihood represented by Expression (20).

Meanwhile, Expression (34) is specifically derived by modifying the following Expressions (36) and (37).

$$\begin{matrix}[ Expression 36 ] & \\ {E}_{q ( {y}_{1 : R} ) q ( {\overline{z}}_{1 : R} )} [ log \frac{p ( {x}_{1 : R} \vert {y}_{1 : R} ) p ( {y}_{1 : R} \vert {\overline{z}}_{1 : R} ) p ( {\overline{z}}_{1 : R} )}{q ( {y}_{1 : R} ) q ( {\overline{z}}_{1 : R} )} ] = {E}_{q ( {y}_{1 : R} )} [ log p ( {x}_{1 : R} \vert {y}_{1 : R} ) + {E}_{q ( {\overline{z}}_{1 : R} )} [ log p ( {y}_{1 : R} \vert {\overline{z}}_{1 : R} ) ] - log q ( {y}_{1 : R} ) ] + {E}_{q ( {\overline{z}}_{1 : R} )} [ log \frac{p ( {\overline{z}}_{1 : R} )}{q ( {\overline{z}}_{1 : R} )} ] = log ρ ( {x}_{1 : R} ) - KL ( q ( {y}_{1 : R} ) \vert \vert ρ ( {y}_{1 : R} \vert {x}_{1 : R} ) ) + {E}_{q ( {\overline{z}}_{1 : R} )} [ log \frac{p ( {\overline{z}}_{1 : R} )}{q ( {\overline{z}}_{1 : R} )} ] \leq log ρ ( {x}_{1 : R} ) + {E}_{q ( {\overline{z}}_{1 : R} )} [ log \frac{p ( {\overline{z}}_{1 : R} )}{q ( {\overline{z}}_{1 : R} )} ] . & ( 36 )\end{matrix}$$

$$\begin{matrix}[ Expression 37 ] & \\ ρ ( {x}_{1 : R} ) = \int p ( {x}_{1 : R} \vert {y}_{1 : R} ) = \int p ( {x}_{1 : R} \vert {y}_{1 : R} ) exp ( {E}_{q ( {\overline{z}}_{1 : R} )} [ log p ( {y}_{1 : R} \vert {\overline{z}}_{1 : R} ) ] ) {dy}_{1 : R} , ρ ( {y}_{1 : R} \vert {x}_{1 : R} ) = \frac{p ( {x}_{1 : R} \vert {y}_{1 : R} ) exp ( {E}_{q ( {\overline{z}}_{1 : R} )} [ log p ( {y}_{1 : R} \vert {\overline{z}}_{1 : R} ) ] )}{\int p ( {x}_{1 : R} \vert {y}_{1 : R} ) exp ( {E}_{q ( {\overline{z}}_{1 : R} )} [ log p ( {y}_{1 : R} \vert {\overline{z}}_{1 : R} ) ] ) {dy}_{1 : R}} & ( 37 )\end{matrix}$$

FIG. 2 is a diagram illustrating an example of a hardware configuration of the learning apparatus 1 in the embodiment. The learning apparatus 1 includes a control unit 11 provided with an processor 91 such as a central processing unit (CPU) and a memory 92 which are connected to each other through a bus, and executes a program. The learning apparatus 1 functions as a device including the control unit 11, an input unit 12, a communication unit 13, a storage unit 14, and an output unit 15 by executing a program.

More specifically, the processor 91 reads out the program stored in the storage unit 14, and stores the read-out program in the memory 92. By the processor 91 executing the program stored in the memory 92, the learning apparatus 1 functions as the device including the control unit 11, the input unit 12, the communication unit 13, the storage unit 14, and the output unit 15.

The control unit 11 controls operations of various functional units included in the learning apparatus 1. The control unit 11 executes a learning process. The control unit 11 controls, for example, an operation of the output unit 15, and causes the output unit 15 to output the execution result of the learning process. The control unit 11 records, for example, various types of information generated by the execution of the learning process in the storage unit 14. Various types of information stored in the storage unit 14 include, for example, the learning result of the linear sum estimation learning model.

The input unit 12 is configured to include an input device such as a mouse, a keyboard, or a touch panel. The input unit 12 may be configured as an interface for connecting the input device to the learning apparatus 1. The input unit 12 accepts inputs of various types of information to the learning apparatus 1. For example, the cardiac sound time series is input to the input unit 12.

The communication unit 13 is configured to include a communication interface for connecting the learning apparatus 1 to an external device. The communication unit 13 communicates with the external device through wired or wireless connection. The external device is, for example, a device which is a transmission source of the cardiac sound time series. The external device is, for example, the analysis apparatus 2. The communication unit 13 transmits the linear sum estimation learning model learned through communication with the analysis apparatus 2 to the analysis apparatus 2.

The storage unit 14 is configured using a computer readable storage medium device such as a magnetic hard disk device or a semiconductor storage device. The storage unit 14 stores various types of information relating to the learning apparatus 1. The storage unit 14 stores, for example, information which is input through the input unit 12 or the communication unit 13. The storage unit 14 stores, for example, the linear sum estimation learning model. The storage unit 14 stores, for example, various types of information generated by the execution of the learning process.

The output unit 15 outputs various types of information. The output unit 15 is configured to include a display device such as, for example, a cathode ray tube (CRT) display, a liquid crystal display, or an organic electro-luminescence (EL) display. The output unit 15 may be configured as an interface for connecting the display device to the learning apparatus 1. The output unit 15 outputs, for example, information which is input to the input unit 12 or the communication unit 13. The output unit 15 may display, for example, the execution result of the learning process.

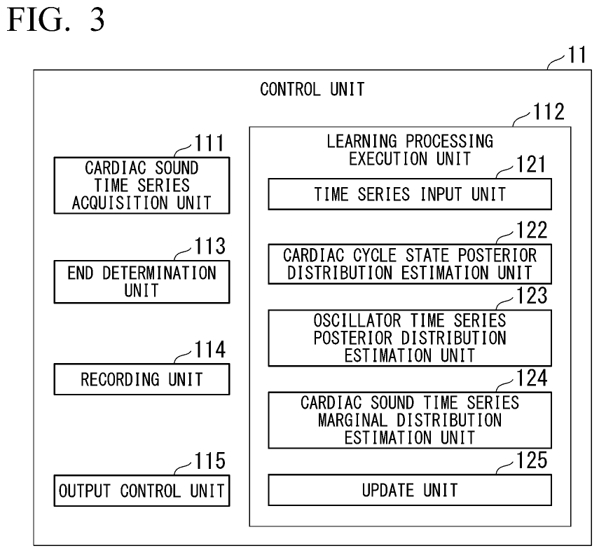

FIG. 3 is a diagram illustrating an example of a configuration of the control unit 11 included in the learning apparatus 1 in the embodiment. The control unit 11 includes a cardiac sound time series acquisition unit 111, a learning processing execution unit 112, an end determination unit 113, a recording unit 114, and an output control unit 115.

The cardiac sound time series acquisition unit 111 acquires the cardiac sound time series which is input to the input unit 12 or the communication unit 13. In a case where the cardiac sound time series has been recorded in the storage unit 14 in advance, the cardiac sound time series acquisition unit 111 may read out the cardiac sound time series from the storage unit 14.

The learning processing execution unit 112 executes the learning process. The end determination unit 113 determines whether the learning end condition is satisfied. The linear sum estimation learning model which is obtained through the learning process executed by the learning processing execution unit 112 and which is the linear sum estimation learning model at a point in time when the learning end condition is satisfied by the end determination unit 113 is the learned linear sum estimation learning model.

The learning processing execution unit 112 includes a time series input unit 121, a cardiac cycle state posterior distribution estimation unit 122, an oscillator time series posterior distribution estimation unit 123, a cardiac sound time series marginal distribution estimation unit 124, and an update unit 125.

The time series input unit 121 executes the time series input process with respect to the cardiac sound time series acquired by the cardiac sound time series acquisition unit 111. That is, the time series input unit 121 inputs the cardiac sound time series acquired by the cardiac sound time series acquisition unit 111 to the linear sum estimation learning model.

The cardiac cycle state posterior distribution estimation unit 122 executes the cardiac cycle state posterior distribution estimation process. The oscillator time series posterior distribution estimation unit 123 executes the oscillator time series posterior distribution estimation process. The cardiac sound time series marginal distribution estimation unit 124 executes the cardiac sound time series marginal distribution estimation process. The update unit 125 executes the update process.

The recording unit 114 records various types of information in the storage unit 14. The output control unit 115 controls the operation of the output unit 15.

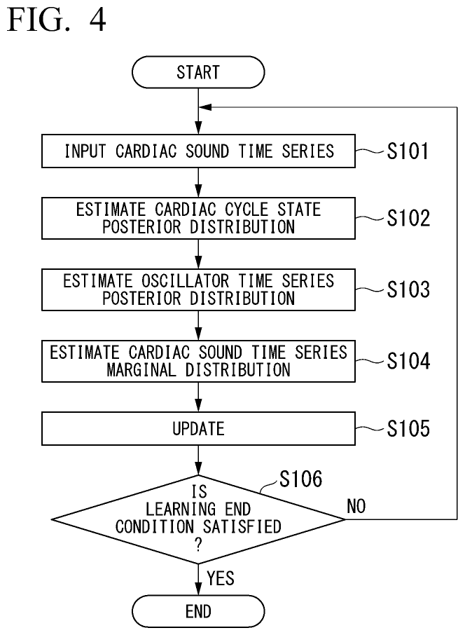

FIG. 4 is a flow chart illustrating an example of a flow of processes which are executed by the learning apparatus 1 in the embodiment. The time series input unit 121 inputs the cardiac sound time series to linear sum estimation learning model (step S101). Next, the cardiac cycle state posterior distribution estimation unit 122 executes the cardiac cycle state posterior distribution estimation process (step S102). That is, the cardiac cycle state posterior distribution estimation unit 122 estimates the posterior distribution of the state of the cardiac cycle on the basis of the input cardiac sound time series.

Next, the oscillator time series posterior distribution estimation unit 123 executes the oscillator time series posterior distribution estimation process (step S103). That is, the oscillator time series posterior distribution estimation unit 123 estimates the posterior distribution of the oscillator time series on the basis of the posterior distribution of the state of the cardiac cycle. Next, the cardiac sound time series marginal distribution estimation unit 124 executes the cardiac sound time series marginal distribution estimation process (step S104). That is, the cardiac sound time series marginal distribution estimation unit 124 estimates the marginal distribution of the cardiac sound time series using the posterior distribution of the oscillator time series. Next, the update unit 125 executes the update process (step S105). The linear sum estimation learning model is updated by executing the update process.

Next, the end determination unit 113 determines whether the learning end condition is satisfied (step S106). In a case where the learning end condition is not satisfied (step S106: NO), the flow returns to the process of step S101. On the other hand, in a case where the learning end condition is satisfied (step S106: YES), the process ends.

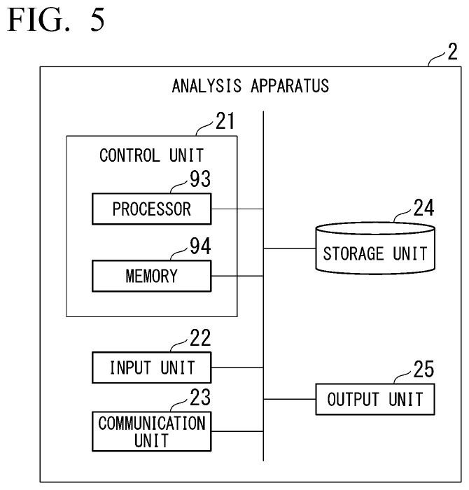

FIG. 5 is a diagram illustrating an example of a hardware configuration of the analysis apparatus 2 in the embodiment. The analysis apparatus 2 includes a control unit 21 provided with a processor 93 such as a CPU and a memory 94 which are connected to each other through a bus, and executes a program. The analysis apparatus 2 functions as a device including the control unit 21, an input unit 22, a communication unit 23, a storage unit 24, and an output unit 25 by executing a program.

More specifically, the processor 93 reads out the program stored in the storage unit 24, and stores the read-out program in the memory 94. By the processor 93 executing the program stored in the memory 94, the analysis apparatus 2 functions as the device including the control unit 21, the input unit 22, the communication unit 23, the storage unit 24, and the output unit 25.

The control unit 21 controls operations of various functional units included in the analysis apparatus 2. The control unit 21 executes the learned linear sum estimation learning model. The control unit 21 controls, for example, an operation of the output unit 25, and causes the output unit 25 to output the execution result of the learned linear sum estimation learning model. The control unit 21 records, for example, various types of information generated by executing the learned linear sum estimation learning model in the storage unit 24.

The input unit 22 is configured to include an input device such as a mouse, a keyboard, or a touch panel. The input unit 22 may be configured as an interface for connecting the input device to the analysis apparatus 2. The input unit 22 accepts inputs of various types of information to the analysis apparatus 2. For example, the cardiac sound time series which is a target for analysis is input to the input unit 22.

The communication unit 23 is configured to include a communication interface for connecting the analysis apparatus 2 to an external device. The communication unit 23 communicates with the external device through wired or wireless connection. The external device is, for example, a device that is a transmission source of the cardiac sound time series which is a target for analysis. The external device is, for example, the learning apparatus 1. The communication unit 23 acquires the linear sum estimation learning model learned through communication with the learning apparatus 1.

The storage unit 24 is configured using a computer readable storage medium device such as a magnetic hard disk device or a semiconductor storage device. The storage unit 24 stores various types of information relating to the analysis apparatus 2. The storage unit 24 stores, for example, information which is input through the input unit 22 or the communication unit 23. The storage unit 24 stores, for example, the learned linear sum estimation learning model. The storage unit 24 stores, for example, various types of information generated by the execution of the learned linear sum estimation learning model.

The output unit 25 outputs various types of information. The output unit 25 is configured to include a display device such as, for example, a CRT display, a liquid crystal display, or an organic EL display. The output unit 25 may be configured as an interface for connecting the display device to the analysis apparatus 2. The output unit 25 outputs, for example, information which is input to the input unit 22 or the communication unit 23. The output unit 25 may display, for example, the execution result of the learned linear sum estimation learning model.

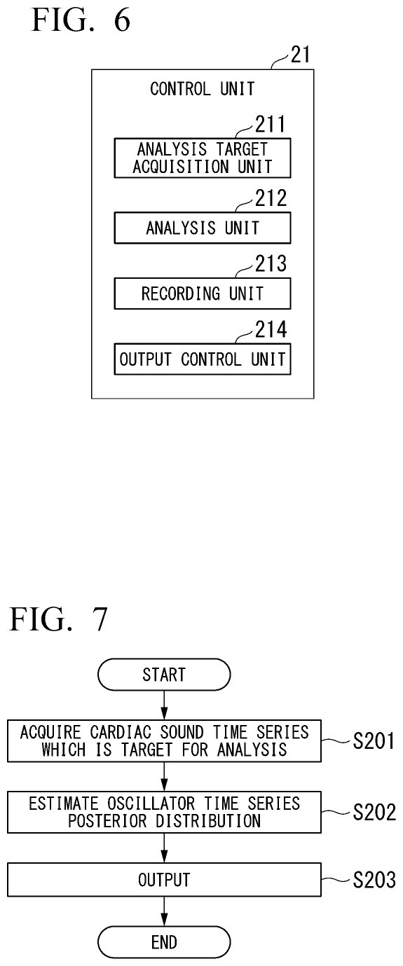

FIG. 6 is a diagram illustrating an example of a configuration of the control unit 21 in the embodiment. The control unit 21 includes an analysis target acquisition unit 211, an analysis unit 212, a recording unit 213, and an output control unit 214.

The analysis target acquisition unit 211 acquires the cardiac sound time series that is a target for analysis which is input to the input unit 22 or the communication unit 23. In a case where the cardiac sound time series which is a target for analysis is recorded in the storage unit 24 in advance, the analysis target acquisition unit 211 may read out the cardiac sound time series which is a target for analysis from the storage unit 24.

The analysis unit 212 analyzes the cardiac sound time series which is a target for analysis. More specifically, the analysis unit 212 executes the learned linear sum estimation learning model with respect to the cardiac sound time series which is a target for analysis to thereby acquire an output of the learned linear sum estimation learning model as the result of analysis. Executing the learned linear sum estimation learning model with respect to the cardiac sound time series which is a target for analysis specifically means inputting the cardiac sound time series which is a target for analysis to the learned linear sum estimation learning model and executing the learned linear sum estimation learning model to which the cardiac sound time series which is a target for analysis is input.

In the execution of the learned linear sum estimation learning model with respect to the cardiac sound time series which is a target for analysis, the posterior distribution of the state of the cardiac cycle is first estimated on the basis of the cardiac sound time series which is a target for analysis. In the execution of the learned linear sum estimation learning model with respect to the cardiac sound time series which is a target for analysis, the posterior distribution of the oscillator time series is next estimated on the basis of the obtained posterior distribution of the state of the cardiac cycle.

The recording unit 213 records various types of information in the storage unit 24. The output control unit 214 controls the operation of the output unit 25.

FIG. 7 is a flow chart illustrating an example of a flow of processes which are executed by the analysis apparatus 2 in the embodiment. The analysis target acquisition unit 211 acquires the cardiac sound time series which is a target for analysis (step S201). Next, the analysis unit 212 executes the learned linear sum estimation learning model with respect to the cardiac sound time series which is a target for analysis (step S202). The posterior distribution of the oscillator time series is estimated by the execution of the learned linear sum estimation learning model. Next, the output control unit 214 causes the output unit 25 to output the obtained posterior distribution of the oscillator time series (step S203).

### <Experiment Results>

The results of experiments using the analysis system 100 will be described. In the experiment, four types of data sets have been used. A first data set and a second data set are normal cardiac sound time series and abnormal cardiac sound time series in various types of symptoms which are associated with auscultation textbooks. These data sets include a total of 119 time series.

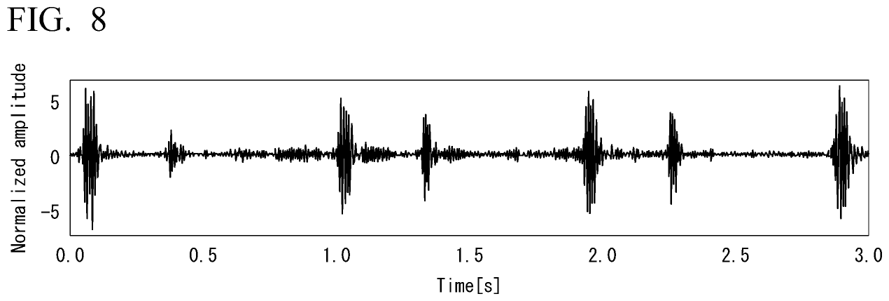

FIG. 8 is a first diagram illustrating an example of a result of an experiment using the analysis system 100 of the embodiment. FIG. 8 is an example of a cardiac sound time series included in the first data set. The vertical axis of FIG. 8 represents a normalized amplitude.

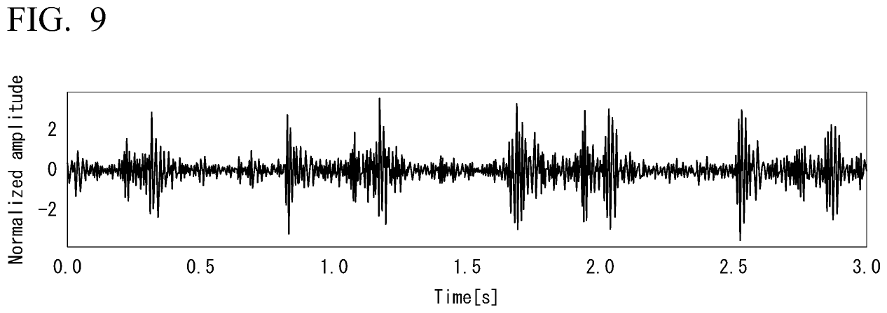

FIG. 9 is a second diagram illustrating an example of a result of an experiment using the analysis system 100 of the embodiment. FIG. 9 is an example of a cardiac sound time series included in the second data set. The vertical axis of FIG. 9 represents a normalized amplitude.

The third data set is a cardiac sound time series obtained by one microphone.

The cardiac sound time series included in the third data set is a cardiac sound time series in which annotations of the S1 sound and the S2 sound are added manually. The fourth data set is a cardiac sound time series obtained simultaneously by a large number of microphones.

In the experiment, a comparison was made with the ensemble empirical mode decomposition method with a kurtosis feature (EEMD) described in Non-Patent Document 9.

In the method using the EEMD, empirical mode decomposition (EMD) was first applied to extract intrinsic mode functions (IMF). During the period between the S1 sound and the S2 sound, the amplitude of each IMF increases simultaneously. In the EEMD, the kurtosis of IMF was calculated using a sliding window in order to detect such an increase. In a case where the window contains the onsets of the S1 sound and the S2 sound, the marginal distribution of the window has a heavier lower edge and a higher kurtosis than in a case where the window does not contain the onsets of the S1 sound and the S2 sound. Therefore, the onsets of the S1 sound and the S2 sound can be estimated by detecting the peak of the product of the kurtosis of the marginal distribution of the IMF in a window having a different scale.

In the experiment, all the cardiac sound time series were downsampled to 2,000 Hz and underwent bandpass filtering through a frequency band from 10 Hz to 150 Hz. The time series thus obtained was applied to the analysis system 100 and the EEMD. In the experiment, the potential of Expression (28) was used as the potential, s was 64, and φ was learned through a two-layer CNN. Although the noise level differs for each data, the same value was used for hyper parameters of both methods in the experiment in order to verify robustness.

It was determined that the estimation of the onset of the S1 sound and the S2 sound was appropriate insofar as an interval between the estimated onset and the true onset is 100 ms.

An F1 score was used to verify the appropriateness of the segmentation. The F1 score is an amount which is defined by the following Expression (38).

$$\begin{matrix}[ Expression 38 ] & \\ [ ( 2 \times {P}_{+} \times {S}_{e} ) / ( {P}_{+} + {S}_{e} ) ] & ( 38 )\end{matrix}$$

P₊ is precision, and Sₑ is recall.

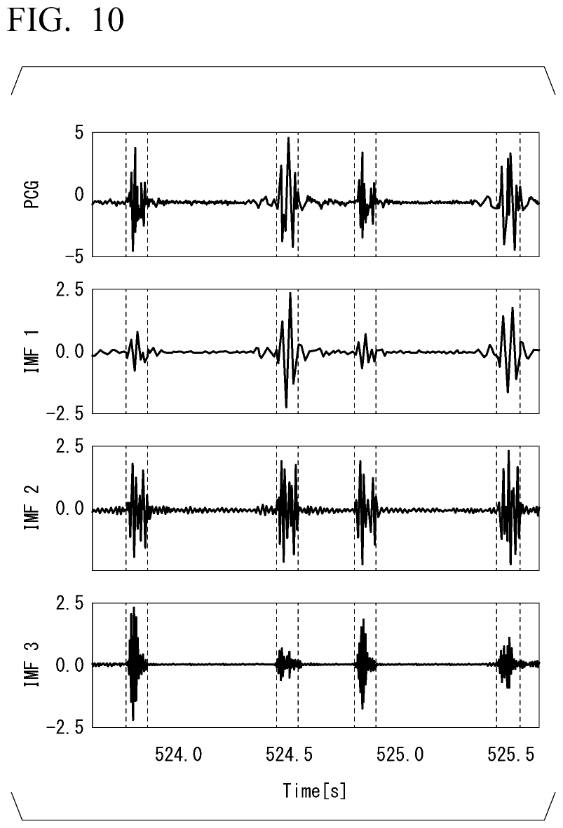

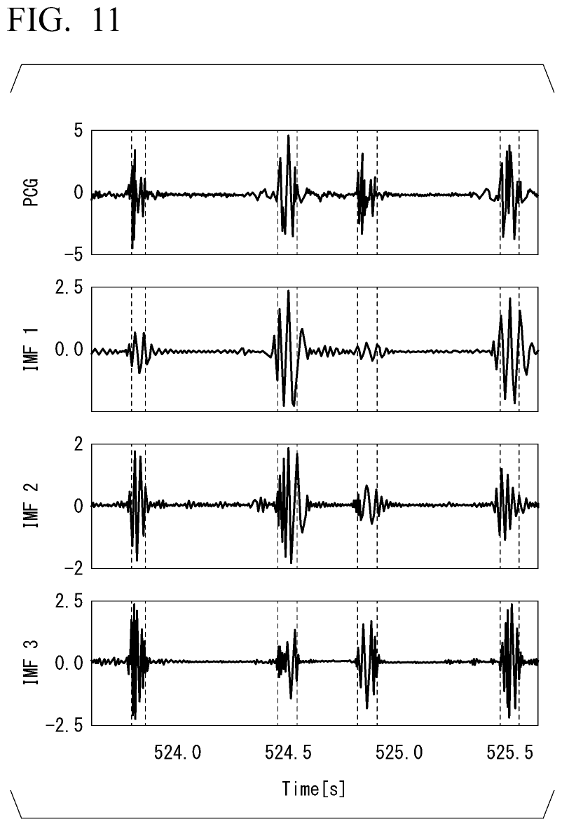

FIG. 10 is a third diagram illustrating an example of a result of an experiment using the analysis system 100 of the embodiment. FIG. 10 is an example of oscillator decomposition obtained from a normal cardiac sound time series using the analysis system 100. The oscillator decomposition involves acquiring the waveform of each fluctuating oscillator of the oscillator linear sum. PCG indicates a cardiac sound time series which is a target for analysis, and each of IMF 1 to IMF 3 indicates a time series of a fluctuating oscillator obtained from the cardiac sound time series which is a target for analysis. The vertical axis of each graph of the PCG, the IMF 1, the IMF 2, and the IMF 3 represents the amplitude. It is true of FIGS. 11 to 13 that the PCG indicates a cardiac sound time series which is a target for analysis, each of IMF 1 to IMF 3 indicates a time series of a fluctuating oscillator obtained from the cardiac sound time series which is a target for analysis, and the vertical axis of each graph of the PCG, the IMF 1, the IMF 2, and the IMF 3 represents the amplitude.

FIG. 11 is a fourth diagram illustrating an example of a result of an experiment using the analysis system 100 of the embodiment. FIG. 11 is an example of oscillator decomposition obtained from a normal cardiac sound time series using the EEMD.

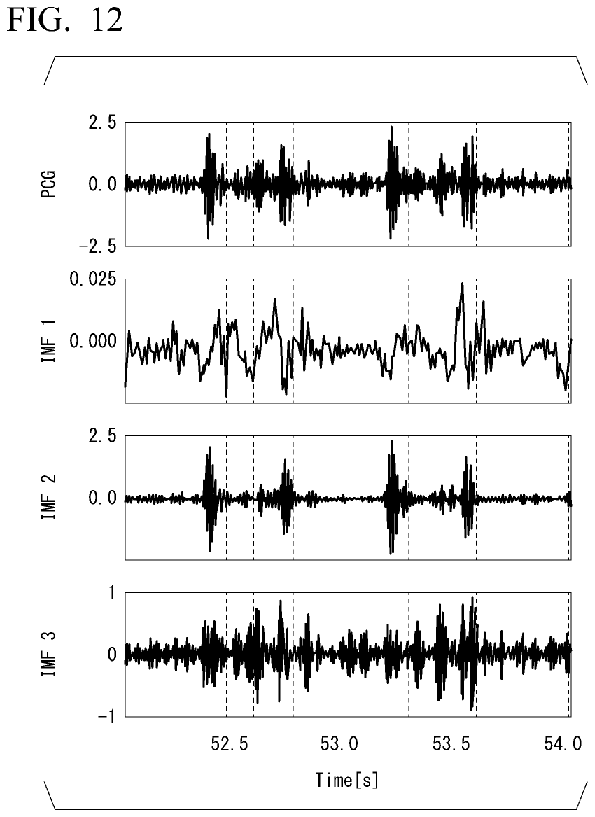

FIG. 12 is a fifth diagram illustrating an example of a result of an experiment using the analysis system 100 of the embodiment. FIG. 12 is an example of oscillator decomposition obtained from an abnormal cardiac sound time series using the analysis system 100.

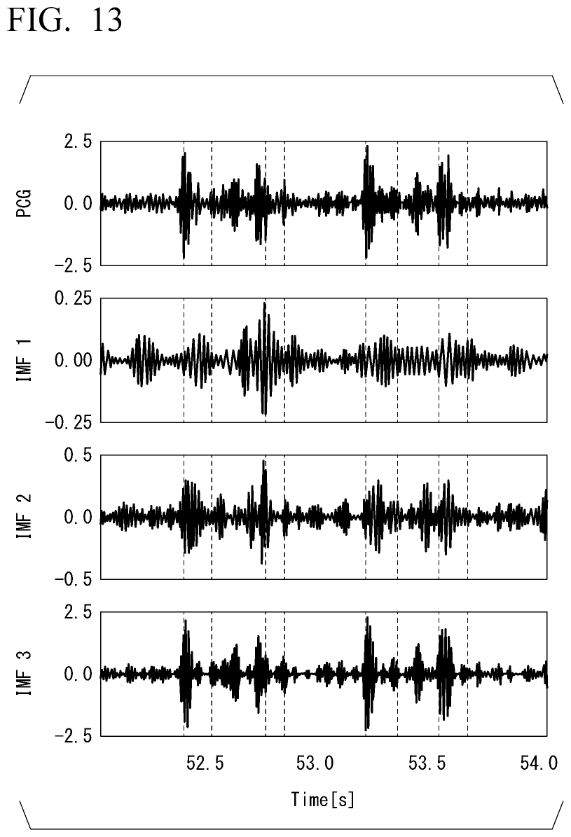

FIG. 13 is a sixth diagram illustrating an example of a result of an experiment using the analysis system 100 of the embodiment. FIG. 13 is an example of oscillator decomposition obtained from an abnormal cardiac sound time series using the EEMD.

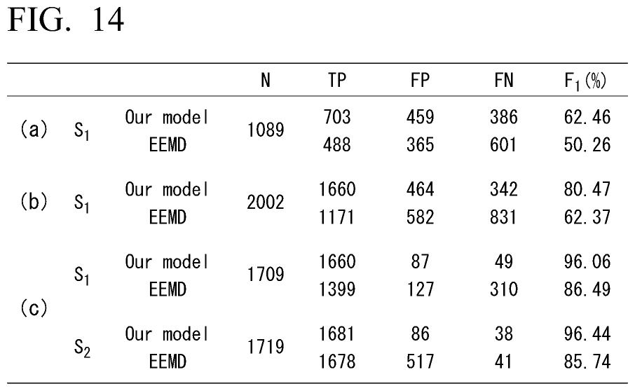

FIG. 14 is a seventh diagram illustrating an example of a result of an experiment using the analysis system 100 of the embodiment. The term “Our model” of FIG. 14 refers to the analysis system 100. “N” of FIG. 14 is the number of S1 sounds and the S2 sounds. “TP” of FIG. 14 is true positive. “FP” of FIG. 14 is false positive. “FN” of FIG. 14 is false negative. “F₁” of FIG. 14 is the F1 score. In addition, (a) of FIG. 14 is the first data set, (b) of FIG. 14 is the second data set, and (c) of FIG. 14 is the third data set.

FIG. 14 shows that the estimation of the onsets of the S1 sound and the S2 sound performed by the analysis system 100 is more accurate than in the EEMD in all three data sets from the first data set to the third data set.

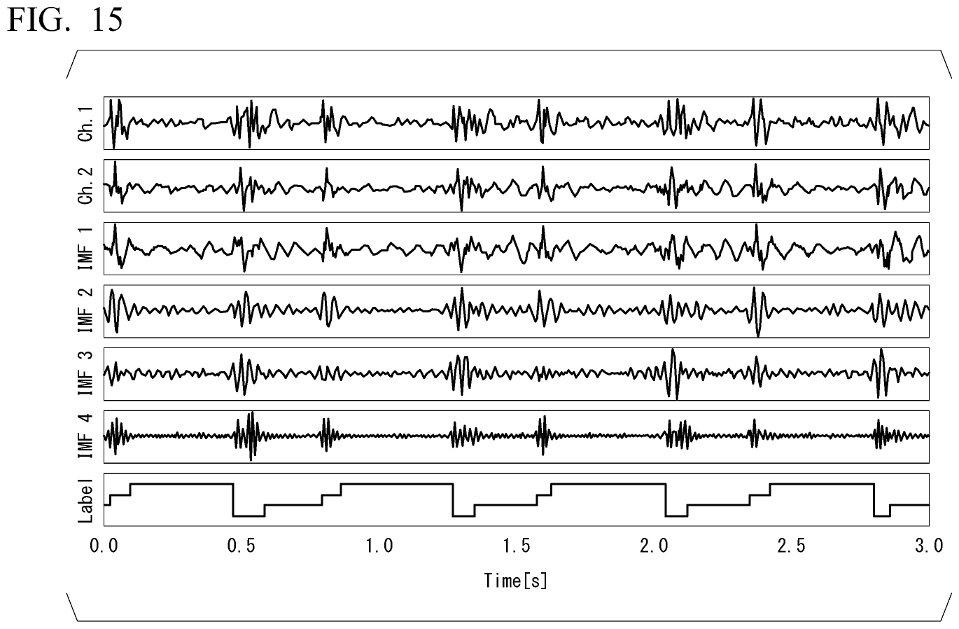

FIG. 15 is an eighth diagram illustrating an example of a result of an experiment using the analysis system 100 of the embodiment. Each of Ch 1 and Ch 2 of FIG. 15 is an oscillator time series which is a target for analysis and indicates a time series of each dimension of a two-dimensional oscillator time series. IMF 1, IMF 2, IMF 3 and IMF 4 of FIG. 15 indicate waveforms of four fluctuating oscillators obtained from the oscillator time series of Ch 1 and Ch 2. The vertical axis of each graph of Ch 1, Ch 2, IMF 1, IMF 2, IMF 3 and IMF 4 represents the amplitude. The graph of Label of FIG. 15 represents the estimated state of the cardiac cycle. FIG. 15 shows that the analysis system 100 can extract a fluctuating oscillator from the cardiac sound time series acquired by a plurality of microphones.

The analysis system 100 of the embodiment configured in this manner obtains a learned linear sum estimation learning model on the basis of an expression representing the generation mechanism of the cardiac sound time series and a mathematical model representing the relationship between a probabilistic state transition and a symbol output. Therefore, it is possible to obtain a linear sum of fluctuating oscillators from the cardiac sound time series which is a target for analysis with a higher level of accuracy than in a mathematical model obtained without using an expression representing the generation mechanism of the cardiac sound time series. Therefore, the analysis system 100 can improve the accuracy of analysis of a time series represented by the linear sum of fluctuating oscillators.

In addition, the analysis system 100 of the embodiment is not a technique of using empirical mode decomposition. In a case where the empirical mode decomposition is used, the occurrence of a problem called Mode mixing has been known. Since the analysis system 100 is not a technique of using the empirical mode decomposition, it can suppress the occurrence of Mode mixing.

In addition, in the technique of empirical mode decomposition, it is difficult to incorporate the generation mechanism of a time series into a mathematical model for obtaining a linear sum of fluctuating oscillators due to heuristic calculation or loose restrictions imposed on the time series after decomposition. Since the analysis system 100 of the embodiment is not a technique of using empirical mode decomposition, as represented by Expression (18), the analysis system 100 can incorporate the generation mechanism of a time series into a mathematical model for obtaining the linear sum of fluctuating oscillators.

In addition, the technique of empirical mode decomposition not being able to obtain a linear sum of fluctuating oscillators from a multi-channel time series recorded simultaneously by a plurality of microphones has become known. The analysis system 100 assumes a situation in which a multi-dimensional time series is observed, and thus can obtain a linear sum of fluctuating oscillators from a multi-channel time series.

### Modification Example

As described above, the cardiac sound time series is an example, and the analysis system 100 does not necessarily have to analyze the time series of the cardiac sound. The time series which is a target for analysis in the analysis system 100 may be any time series insofar as it is a time series represented by a linear sum of amplitude waveforms of the fluctuating oscillator. The time series represented by a linear sum of amplitude waveforms of the fluctuating oscillator may be, for example, a time series of a respiratory sound. In a case where the time series represented by a linear sum of amplitude waveforms of the fluctuating oscillator is a time series of a respiratory sound, the analysis system 100 uses an expression representing the generation mechanism of the respiratory sound time series instead of an expression representing the generation mechanism of the cardiac sound time series. In this manner, the expression representing the generation mechanism of the cardiac sound time series is an example of an expression representing the generation mechanism of a time series which is a target for analysis.

In addition, the state of a mathematical model representing the relationship between a probabilistic state transition and a symbol output is a two-phase state of an expiratory phase and an inspiratory phase in a case where the time series represented by a linear sum of amplitude waveforms of the fluctuating oscillator is a respiratory sound. In this manner, the state of a mathematical model representing the relationship between a probabilistic state transition and a symbol output is the state of a generation source of a time series represented by a linear sum of amplitude waveforms of the fluctuating oscillator.

The learning apparatus 1 and the analysis apparatus 2 may be mounted using a plurality of information processing devices which are communicably connected to each other through a network. In this case, each functional unit included in the learning apparatus 1 and the analysis apparatus 2 may be distributed and mounted in a plurality of information processing devices.

Meanwhile, the learning apparatus 1 and the analysis apparatus 2 do not necessarily have to be mounted as different devices. The learning apparatus 1 and the analysis apparatus 2 may be mounted as, for example, one device having both functions.

Meanwhile, all or some of the functions of the analysis system 100, the learning apparatus 1, and the analysis apparatus 2 may be realized using hardware such as an application specific integrated circuit (ASIC), a programmable logic device (PLD), a field programmable gate array (FPGA), or the like. The program may be recorded in a computer readable recording medium. The computer readable recording medium refers to, for example, a portable medium such as a flexible disk, a magnetooptic disc, a ROM, or a CD-ROM, or a storage device such as a hard disk built into a computer system. The program may be transmitted through an electrical telecommunication line.

Meanwhile, the cardiac sound time series is an example of an observed time series.

Hereinbefore, the embodiments of the present invention have been described in detail with the accompanying drawings, but specific configurations are not limited to these embodiments, and may also include a design and the like without departing from the scope of the present invention.

### REFERENCE SIGNS LIST

100 Analysis system
1 Learning apparatus
2 Analysis apparatus
11 Control unit
12 Input unit
13 Communication unit
14 Storage unit
15 Output unit
111 Cardiac sound time series acquisition unit
112 Learning processing execution unit
113 End determination unit
114 Recording unit
115 Output control unit
121 Time series input unit
122 Cardiac cycle state posterior distribution estimation unit
123 Oscillator time series posterior distribution estimation unit
124 Cardiac sound time series marginal distribution estimation unit
125 Update unit
21 Control unit
22 Input unit
23 Communication unit
24 Storage unit
25 Output unit
211 Analysis target acquisition unit
212 Analysis unit
213 Recording unit
214 Output control unit
91 Processor
92 Memory
93 Processor
94 Memory

1. A learning apparatus comprising:
a processor; and
a storage medium having computer program instructions stored thereon, wherein the computer program instruction, when executed by the processor, perform processing of:
with a time series of an amplitude of a fluctuating oscillator whose amplitude changes periodically being defined as an oscillator time series, acquiring an observed time series which is a time series represented by an oscillator linear sum which is a linear sum of the oscillator time series; and
using an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series,
wherein the linear sum estimation learning model on the basis of a result of execution of the linear sum estimation learning model is updated.

2. The learning apparatus according to claim 1, wherein the mathematical model representing a relationship between a probabilistic state transition of a state of a generation source of the observed time series and a symbol output which is information probabilistically output in the state is a hidden semi-Markov model.

3. The learning apparatus according to claim 1 wherein the observed time series is a time series of a cardiac sound.

4. An analysis apparatus comprising:
a processor; and
a storage medium having computer program instructions stored thereon, wherein the computer program instruction, when executed by the processor, perform processing of:
acquiring a time series which is a target for analysis; and
analyzing a time series which is a target for analysis using a learned linear sum estimation learning model obtained by a learning apparatus comprising a processor; and a storage medium having computer program instructions stored thereon, wherein the computer program instruction, when executed by the processor, perform processing of, with a time series of an amplitude of a fluctuating oscillator whose amplitude changes periodically being defined as an oscillator time series, acquiring an observed time series which is a time series represented by an oscillator linear sum which is a linear sum of the oscillator time series; and using an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, wherein the linear sum estimation learning model on the basis of a result of execution of the linear sum estimation learning model is updated.

5. A learning method comprising:
with a time series of an amplitude of a fluctuating oscillator whose amplitude changes periodically being defined as an oscillator time series, acquiring an observed time series which is a time series represented by an oscillator linear sum which is a linear sum of the oscillator time series; and
using an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series,
wherein the linear sum estimation learning model on the basis of a result of execution of the linear sum estimation learning model is updated.

6. An analysis method comprising:
acquiring a time series which is a target for analysis; and
analyzing a time series which is a target for analysis using a learned linear sum estimation learning model obtained by a learning apparatus comprising a processor; and a storage medium having computer program instructions stored thereon, wherein the computer program instruction, when executed by the processor, perform processing of, with a time series of an amplitude of a fluctuating oscillator whose amplitude changes periodically being defined as an oscillator time series, acquiring an observed time series which is a time series represented by an oscillator linear sum which is a linear sum of the oscillator time series; and using an expression representing a generation mechanism of the observed time series and a mathematical model representing a relationship between a probabilistic state transition of a state of a generation source of the observed time series and a symbol output which is information probabilistically output in the state to execute a linear sum estimation learning model which is a mathematical model that is configured to estimate the oscillator linear sum of the observed time series on the basis of the observed time series, wherein the linear sum estimation learning model on the basis of a result of execution of the linear sum estimation learning model is updated.

7. A non-transitory computer-readable medium having computer-executable instructions that, upon execution of the instructions by a processor of a computer, cause the computer to function as the learning apparatus according to claim 1.

8. A non-transitory computer-readable medium having computer-executable instructions that, upon execution of the instructions by a processor of a computer, cause the computer to function as the analysis apparatus according to claim 4.
